(19) United States
(12) Patent Application Publication
(10) Pub. No.: US 2009/0209581 A1
(43) Pub. Date: Aug. 20, 2009

(54) NITROXIDES FOR USE IN TREATING OR PREVENTING IMMUNOLOGICAL DISEASES

(76) Inventors: Louis Habash, Newport Beach, CA (US); Clarence Jones, Huntington Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/815,441

(22) PCT Filed: Feb. 2, 2006
(86) PCT No.: PCT/US06/03973

§ 371 Date: Jun. 30, 2008

Related U.S. Application Data
(60) Provisional application No. 60/649,252, filed on Feb. 2, 2005.

(51) Int. Cl. A61K 31/445; C07D 211/00; A61P 37/00

(52) U.S. Cl. 514/315; 546/242

(57) ABSTRACT

Pharmaceutical compositions are provided that are useful in treating immunological diseases. The compositions comprise a pharmaceutically acceptable carrier, and an effective therapeutic or prophylactic amount of a nitroxide antioxidant that alters the expression of a gene related to an immunological disease. Methods are also provided for the use of the pharmaceutical compositions in the treatment or prevention of immunological disease. In a preferred embodiment, the nitroxide antioxidant is Tempol (4-hydroxy-2,2,6,6-tetramethylpiperidine-1-oxyl); the gene is one of glucocorticoid-induced leucine zipper, collagen type I alpha 1, and complement component 6; and the immunological disease is Graves' disease, Hashimoto's thyroiditis, autoimmune polyglandular syndrome, insulin-dependent diabetes mellitus, insulin-independent diabetes mellitus, immune-mediated infertility, autoimmune Addison's disease, pemphigus vulgaris, pemphigus foliaceus, dermatitis herpetiformus, vitiligo, autoimmune hemolytic anemia, autoimmune thrombocytopenic purpura, pernicious anemia, myasthenia gravis, multiple sclerosis, Guillain-Barre syndrome, stiff-man syndrome, acute rheumatic fever, sympathetic ophthalmia, Goodpasture's syndrome, systemic lupus erythematosus, rheumatoid arthritis, systemic necrotizing vasculitis, Wegener's granulomatosis, antiphospholipid syndrome, Sjögren's syndrome, scleroderma, accelerated graft arteriosclerosis, graft-versus-host disease, Crohn's disease, or tuberculosis.

### BACKGROUND OF THE INVENTION

1. Field of the Invention

The present invention relates to pharmaceutical compositions useful for treating or preventing immunological diseases such as autoimmune disease, and to methods for using these compositions in treating or preventing such diseases.

2. Description of the Related Art

The human immune system has evolved over millions of years to develop defense mechanisms that are highly specific for invading pathogens. It has two main branches: the innate immune system, which is the more ancient and can be traced to invertebrates, and the adaptive immune system, which is found only in vertebrates.

The innate immune system uses germ-line encoded proteins to recognize molecular features common to microbes. Macrophages, one type of cell involved in this branch of the immune system, recognize these features (termed PAMPs) using surface receptor molecules. Once the macrophage recognizes a pathogen, it engulfs and destroys it. Macrophages activated by this process secrete cytokines and chemokines that result in vasodilation, increased vascular permeability, and the attraction of inflammatory cells, such as neutrophils, which participate in the destruction of the invading microbes at the site of infection. Other monocytes are also attracted to the site and rapidly differentiate to become macrophages. It is this process that produces the characteristic signs of inflammation: heat, pain, redness, and swelling. Inflammation can also be triggered by the complement cascade.

The inflammation produced by an innate immune response initiated by activated macrophages also contributes to the initiation of an adaptive immune response, by increasing the flow of lymph containing antigen and antigen-presenting cells into lymphoid tissue. The adaptive immune response can also be initiated by macrophages, but is most often started when an immature dendritic cell in infected tissue engulfs a pathogen. These dendritic cells function to carry pathogen antigens to lymphoid tissue and present them to T lymphocytes, leading to the mounting of an adaptive immune response. As in innate immunity, macrophages are a key end effector of adaptive immunity: they can be activated by certain armed T cells to destroy invading pathogens. Macrophages are thus a key link in immune responses, whether innate or adaptive.

The fine balance of the human immune system can often be upset, however, leading to inappropriate or exaggerated host immune responses. One way in which this can occur is by a mistaken recognition of self antigens as non-self antigens, leading to an autoimmune response. This can occur in a number of ways: exogenous stimuli such as molecular mimicry by certain pathogens, superantigenic stimulation, the failure to eliminate immune cells that recognize self antigens, or injury-related exposure of normally sequestered self antigens, such as those in the brain or the eye. However the autoimmune response is stimulated, it can have potentially devastating consequences, such as widespread destruction of normal body tissues, and can, depending on the severity of the response and the affected tissues, lead to death.

It would be desirable to avoid these consequences by developing methods of suppressing the inappropriate immune system reactions that give rise to them. One potential way to do so is to use genetic therapy to reduce the immune system response, such as by targeting the macrophages that are common end effectors to both branches of the immune system. To this end, it would be desirable to identify genes related to the immune response and to develop methods of altering the expression patterns of those genes so as to prevent the development of immunological disease or reduce its effects once it has occurred.

### SUMMARY OF THE INVENTION

Pharmaceutical compositions are provided that are useful in preventing and treating immunological diseases such as autoimmune disease. The compositions comprise a pharmaceutically acceptable carrier, and an effective therapeutic or prophylactic amount of an agent that changes the expression pattern of a gene related to the diseases. Methods are also provided for the use of the pharmaceutical compositions in the alteration of intracellular levels of immunological disease-related proteins. In a preferred embodiment, the agent is a nitroxide antioxidant, such as Tempol (4-hydroxy-2,2,6,6-tetramethylpiperidine-1-oxyl).

### DETAILED DESCRIPTION OF THE PREFERRED EMBODIMENT

As described above, a composition and method are disclosed which are useful in treating or preventing immunological diseases such as autoimmune disease. As used in this patent, an “immunological disease” is one that involves inappropriate or exaggerated host immunological reactions. In a preferred embodiment, the agent used to change the expression pattern of a gene related to these diseases is a nitroxide antioxidant. Tempol is a stable nitroxide radical characterized by the chemical formula 4-hydroxy-2,2,6,6-tetramethylpiperidine-1-oxyl that has antioxidative properties. The present applicants have discovered that in addition, Tempol also possesses the novel property of altering the expression of genes encoding for proteins associated with immunological diseases such as autoimmune disease (see Tables 1-2 below). Previous therapies have generally not focused on altering the expression patterns of such immunological disease-related genes.

The use of other nitroxide compounds is also contemplated. According to certain embodiments the nitroxide compound can be selected from the following formulas:

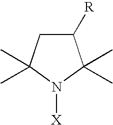

Wherein X is selected from O. and OH, and R is selected from COOH, CONH, CN, and CH₂NH₂.

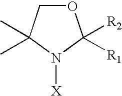

Wherein X is selected from O. and OH, and R₁ is selected from CH₃ and spirocyclohexyl, and R₂ is selected from C₂H₅ and spirocyclohexyl.

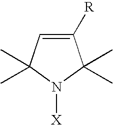

Wherein X is selected from O. and OH and R is selected from CONH.

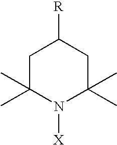

Wherein X is selected from O. and OH and R is selected from H, OH, and NH2.

Suitable nitroxide compounds can also be found in Proctor, U.S. Pat. No. 5,352,442, and Mitchell et al., U.S. Pat. No. 5,462,946, both of which are hereby incorporated by reference in their entireties.

A non-limiting list of nitroxide compounds include: 2-ethyl-2,5,5-trimethyl-3-oxazolidine-1-oxyl (OXANO), 2,2,6,6-tetramethylpiperidine-1-oxyl (TEMPO), 4-hydroxy-2,2,6,6-tetramethylpiperidine-1-oxyl (TEMPOL), 4-amino-2,2,6,6-tetramethlyl-1-piperidinyloxy (Tempamine), 3-Aminomethyl-PROXYL, 3-Cyano-PROXYL, 3-Carbamoyl-PROXYL, 3-Carboxy-PROXYL, and 4-Oxo-TEMPO. TEMPO can also be substituted, typically in the 4 position, for example, 4-amino, 4-(2-bromoacetamido), 4-(ethoxyfluorophosphonyloxy), 4-hydroxy, 4-(2-iodoacetamido), 4-isothiocyanato, 4-maleimido, 4-(4-nitrobenzoyloxyl), 4-phosphonooxy, and the like.

### Experimental Protocols

To assess the effects of Tempol on gene expression, Tempol was administered to experimental mice at a dose of 5 mg/g of food from 14 months to 31 months after birth. Mice receiving the same food without the addition of Tempol were used as a negative control. At the age of 31 months, the experimental animals were sacrificed and the hearts were surgically removed. The expression of a broad spectrum of genes in the cardiac tissue was assessed using chip-based microarray technology. Such chips are well known in the art and are widely used to assess gene expression. The experimental results showed that a gene related to immunological disease, glucocorticoid-induced leucine zipper (GILZ), exhibited a more than twofold increase in expression. This gene is shown in Table 1.

TABLE 1
AUTOIMMUNE DISEASE-RELATED GENE EXIBITING INCREASED EXPRESSION
IN CARDIAC TISSUE AFTER TEMPOL ADMINISTRATION

| | | Control mice | | | TEMPOL-treated mice | | | |
|---|---|---|---|---|---|---|---|---|
| ORF | Description | tpc1 | tpc2 | tpc3 | tp51 | tp52 | tp53 | Fold change |

UPREGULATED GENE

| | | | | | | | | |
|---|---|---|---|---|---|---|---|---|
| AA097711 | Glucocorticoid- | 21 | −31 | −16 | 83 | 33 | 4 | 2.3 |
| | induced leucine | | | | | | | |
| | zipper | | | | | | | |

In a further gene expression study, Tempol was administered to experimental mice at a dose of 5 g/kg of diet from 12 months through 15 months. Mice receiving the same diet without the addition of Tempol were used as a negative control. At the age of 15 months, the adipose tissue of the experimental animals was obtained. The expression of a broad spectrum of genes in the adipose tissue was assessed using chip-based microarray technology. Specifically, in this case an Affymetrix MOE430A 2.0 array, containing 12,960 genes, was employed. Such chips are well known in the art and are widely used to assess gene expression. The experimental results showed that a gene related to the autoimmune disease scleroderma, and which is overexpressed in scleroderma patients, collagen type I alpha 1 (COL1A1), exhibited a decrease in expression. Furthermore, a gene involved in the complement cascade, complement component 6, a decreased level of which has been shown to be associated with prolonged survival of allographs and the attenuation of multiple sclerosis in an animal model, exhibited a decrease in expression. These genes are shown in Table 2.

TABLE 2
AUTOIMMUNE DISEASE-RELATED GENES EXIBITING
DECREASED EXPRESSION IN ADIPOSE TISSUE
AFTER TEMPOL ADMINISTRATION

| | Mean | Mean (Tempol- | P | Fold |
|---|---|---|---|---|
| Description | (Control mice) | treated mice) | Value | change |
| Collagen | 2153 | 1688 | 0.023 | −1.28 |
| type I alpha | | | | |
| 1 (COL1A1) | | | | |
| Complement | 626 | 337 | 0.001 | −1.85 |
| Component | | | | |
| 6 (C6) | | | | |

A short summary of the genes described in Tables 1 and 2 is provided below.

### Glucocorticoid-Induced Leucine Zipper (GILZ)

GILZ is a newly identified member of the leucine zipper family which is expressed in the lymphocytes of healthy mice and human lymphohemopoietic cells, and has been shown to interfere with the function of two transcriptional activators: activator protein 1 (AP-1) and nuclear factor κβ (NFκβ). Both AP-1 and NFκβ play a critical role in activating macrophages in mobilizing immune reactions. A recent study demonstrated that GILZ is constitutively produced by murine and human macrophages, with production being enhanced by both glucocorticoids and interleukin-10 (L-10), two naturally-occurring immunosuppressive agents. (Berrebi et al., Blood 101(2): 729-738 (2003)) GILZ was shown to bind to a subunit of NFκβ and prevent transcription from NFκβ-dependent regulatory elements, and increased expression of the GILZ gene in monocytes was shown to reproduce the effects of immunosuppressive glucocorticoids and IL-10 (i.e., inhibition of the production of inflammatory chemokines of the costimulatory molecules CD80 and CD 86, and of Toll-like receptor 2, which triggers the NFκβ pathway). Because GILZ mediates the suppression of macrophages, the protein will be useful in the treatment of diseases characterized by inappropriate or exaggerated host immunological reactions, such as autoimmune diseases and allograft rejection and the like.

As shown in Table 1, the expression of GILZ in the cardiac tissue of the experimental mice was increased 2.3-fold in the animals treated with Tempol.

### Collagen Type I, Alpha I (COL1A1)

Collagen type I is present in most connective tissues, the only collagen component in cartilage, and is the major protein of bone matrix. It has been shown to be upregulated in fibroblasts of patients suffering from scleroderma (also known as systemic sclerosis), an illness which is characterized by thickening of the skin as the result of the accumulation of connective tissue and which involves the visceral organs, including the gastrointestinal tract, lungs, heart, and kidneys. Furthermore, there is evidence that cell mediated immunity plays a role in the development of the fibrosis associated with scleroderma. A recent study has shown that the expression level of COL1A1 expressed in fibroblasts obtained from scleroderma patients was downregulated by an anti-neoplastic drug (Louneva, et al., Journal of Biological Chemistry, 278 (41), pages 40400-40407 (2003)). The treated fibroblasts demonstrated a 54% downregulation of COL1A1 transcription, which would be expected to lead to a decrease in the pathogenic fibrosis characterizing the disease. Because COL1A1 is overexpressed in scleroderma, an agent which causes a decrease in the expression level of the gene would be expected to have a beneficial effect.

As shown in Table 2, the expression of COL1A1 in the adipose tissue of the experimental mice was decreased 1.28-fold in the animals treated with Tempol.

### Complement Component 6 (C6)

C6 is one of five plasma proteins that are incorporated into the lytic terminal complex on lipid membranes following activation of the complement cascade. Recent studies in rats have shown that hearts transplanted into major histocompatibility complex-incompatible animals demonstrated significantly longer survival when the recipient animals were deficient in C6. Furthermore, a reduction in accelerated graft arteriosclerosis was also demonstrated in the C6-deficient rats. Furthermore, experimental allergic encephalomyelitis (an animal model of multiple sclerosis) in C6-deficient rats was shown to be significantly milder than that in normal rats following immunization with myelin basic protein (Tran et al., Journal of Immunology 168 (2002) 4293-300; Ota et al., Immunobiology 79:3 (2005) 276-291; Qian et al., Transplantation 72:5 (2001) 900-906). These studies indicate that a reduced level of C6 may lead to a reduced activity of the membrane attack complex, and such a reduction in C6 levels will be useful in the treatment of diseases characterized by inappropriate or exaggerated immunological reactions, such as autoimmune diseases and allograft rejection and the like.

As shown in Table 2, the expression of C6 in the adipose tissue of the experimental mice was decreased 1.85-fold in the animals treated with Tempol.

### Preferred Embodiment

### Autoimmune Disease Prophylaxis and Treatment Protocol

As described above, Tempol has the effect of altering the expression of genes related to immunological reactions. Since the expression is altered, administration of Tempol will have a beneficial effect by changing concentrations of gene products in such a way as to be beneficial in suppressing inappropriate immune system responses. In a preferred embodiment of the present invention, therefore, Tempol is administered to a mammalian host, such as a human, exhibiting no symptoms of an immunological disease such as autoimmune disease in order to prevent the development of the disease. Particularly preferred patients are those who are predisposed or otherwise at risk for immunological diseases, such as those with a family history of autoimmune disease, those with genetic or serum markers associated with autoimmune disease, those exposed to substances known to provoke an autoimmune disease, or those who have undergone an allograft that would be expected to provoke an immune response. Alternatively, Tempol may be administered to a human exhibiting an immunological disease in order to ameliorate the effects of the disease on the patient. For this purpose, Tempol, non-toxic salts thereof, acid addition salts thereof or hydrates thereof may be administered systemically or locally, usually by oral or parenteral administration.

The specific diseases for which prophylaxis or treatment is contemplated include, for example, Graves' disease, Hashimoto's thyroiditis, autoimmune polyglandular syndrome, insulin-dependent diabetes mellitus, insulin-independent diabetes mellitus, immune-mediated infertility, autoimmune Addison's disease, pemphigus vulgaris, pemphigus foliaceus, dermatitis herpetiformus, vitiligo, autoimmune hemolytic anemia, autoimmune thrombocytopenic purpura, pernicious anemia, myasthenia gravis, multiple sclerosis, Guillain-Barre syndrome, scleroderma, accelerated graft arteriosclerosis, stiff-man syndrome, acute rheumatic fever, sympathetic ophthalmia, Goodpasture's syndrome, systemic lupus erythematosus, rheumatoid arthritis, systemic necrotizing vasculitis, Wegener's granulomatosis, antiphospholipid syndrome, Sjögren's syndrome, graft-versus-host disease, and diseases involving delayed type hypersensitivity reactions such as Crohn's disease and tuberculosis.

The doses to be administered are determined depending upon, for example, age, body weight, symptom, the desired therapeutic effect, the route of administration, and the duration of the treatment. In the human adult, the dose per person at a time is generally from about 0.01 to about 1000 mg, by oral administration, up to several times per day. Specific examples of particular amounts contemplated via oral administration include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000 or more mg. The dose per person at a time is generally from about 0.01 to about 100 mg/kg via parenteral administration (preferably intravenous administration), up to several times per day, Specific examples of particular amounts contemplated include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 or more mg/kg. Continuous intravenous administration is also contemplated for from 1 to 24 hours per day to achieve a target concentration from about 0.01 mg/L to about 100 mg/L. Specific examples of particular amounts contemplated via this route include about 0.02, 0.03, 0.04, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more mg/L. The dose to be used does, however, depend upon various conditions, and there may be cases wherein doses lower than or greater than the ranges specified above are used.

Tempol may be administered in the form of, for example, solid compositions, liquid compositions or other compositions for oral administration, injections, liniments or suppositories for parenteral administration.

Solid compositions for oral administration include compressed tablets, pills, capsules, dispersible powders and granules. Capsules include hard capsules and soft capsules. hi such solid compositions, Tempol may be admixed with an excipient (e.g. lactose, mannitol, glucose, microcrystalline cellulose, starch), combining agents (hydroxypropyl cellulose, polyvinyl pyrrolidone or magnesium metasilicate aluminate), disintegrating agents (e.g. cellulose calcium glycolate), lubricating agents (e.g. magnesium stearate), stabilizing agents, agents to assist dissolution (e.g. glutamic acid or aspartic acid), or the like. The agents may, if desired, be coated with coating agents (e.g. sugar, gelatin, hydroxypropyl cellulose or hydroxypropylmethyl cellulose phthalate), or be coated with two or more films. Further, coating may include containment within capsules of absorbable materials such as gelatin.

Liquid compositions for oral administration include pharmaceutically acceptable solutions, suspensions, emulsions, syrups and elixirs. In such compositions, Tempol is dissolved, suspended or emulsified in a commonly used diluent (e.g. purified water, ethanol or mixture thereof). Furthermore, such liquid compositions may also comprise wetting agents or suspending agents, emulsifying agents, sweetening agents, flavoring agents, perfuming agents, preserving agents, buffer agents, or the like.

Injections for parenteral administration include solutions, suspensions, emulsions and solids which are dissolved or suspended. In injections, Tempol may be dissolved, suspended and emulsified in a solvent. The solvents are, for example, distilled water for injection, physiological salt solution, vegetable oil, propylene glycol, polyethylene glycol, alcohol such as ethanol, or a mixture thereof. Moreover the injections may also include stabilizing agents, agents to assist dissolution (e.g. glutamic acid, aspartic acid or POLYSORBATE80 (registered trade mark)), suspending agents, emulsifying agents, soothing agents, buffer agents, preserving agents, etc. They are sterilized in the final process or manufactured and prepared by sterile procedure. They may also be manufactured in the form of sterile solid compositions, such as a freeze-dried composition, and they may be sterilized or dissolved immediately before use in sterile distilled water for injection or some other solvent.

Other compositions for parenteral administration include liquids for external use, and ointment, endermic liniments, inhale, spray, suppositories for rectal administration and pessaries for vaginal administration which comprise Tempol and are administered by methods known in the art.

Spray compositions may comprise additional substances other than diluents: e.g. stabilizing agents (e.g. sodium sulfite hydride), isotonic buffers (e.g. sodium chloride, sodium citrate or citric acid). For preparation of such spray compositions, for example, the method described in U.S. Pat. No. 2,868,691 or No. 3,095,355 may be used. Briefly, a small aerosol particle size useful for effective distribution of the medicament may be obtained by employing self-propelling compositions containing the drugs in micronized form dispersed in a propellant composition. Effective dispersion of the finely divided drug particles may be accomplished with the use of very small quantities of a suspending agent, present as a coating on the micronized drug particles. Evaporation of the propellant from the aerosol particles after spraying from the aerosol container leaves finely divided drug particles coated with a fine film of the suspending agent. In the micronized form, the average particle size is less than about 5 microns. The propellant composition may employ, as the suspending agent, a fatty alcohol such as oleyl alcohol. The minimum quantity of suspending agent is approximately 0.1 to 0.2 percent by weight of the total composition. The amount of suspending agent is preferably less than about 4 percent by weight of the total composition to maintain an upper particle size limit of less than 10 microns, and preferably 5 microns. Propellants that may be employed include hydrofluoroalkane propellants and chlorofluorocarbon propellants. Dry powder inhalation may also be employed.

### Example 1

A 70-kilogram patient diagnosed with an autoimmune disease is administered a dose of 1500 mg of Tempol per day for 180 days. This may be administered in a single dose, or may be administered as a number of smaller doses over a 24-hour period: for example, three 500-mg doses at eight-hour intervals. Following treatment, the protein level of glucocorticoid-induced leucine zipper in circulating monocytes is increased, and the protein levels of complement component 6 in plasma and of collagen type 1 A1 in fibroblasts is reduced.

### Example 2

A 70-kilogram patient is administered a dose of 1500 mg of Tempol per day for 180 days prior to receiving an organ transplant. This may be administered in a single dose, or may be administered as a number of smaller doses over a 24-hour period: for example, three 500-mg doses at eight-hour intervals. Following treatment, the protein level of glucocorticoid-induced leucine zipper in circulating monocytes is increased, and the protein levels of complement component 6 in plasma and of collagen type 1 A1 in fibroblasts is reduced.

What is claimed is:

1. A method for altering intracellular levels of one or more proteins associated with an immunological disease, comprising:
identifying an individual in need of altering levels of immunological disease-associated proteins; and
administering to that individual an effective amount of a nitroxide antioxidant.

2. The method of claim 1, wherein the level of the immunological disease-associated proteins is increased.

3. The method of claim 2, wherein the immunological disease-associated protein is glucocorticoid-induced leucine zipper.

4. The method of claim 1, wherein the level of the immunological disease-associated proteins is decreased.

5. The method of claim 4, wherein the immunological disease-associated protein is selected from the group consisting of collagen type 1A1 and complement component 6.

6. The method of claim 1, wherein the nitroxide antioxidant is 4-hydroxy-2,2,6,6-tetramethylpiperidine-1-oxyl.

7. The method of claim 1, wherein the immunological disease is selected from the group consisting of Graves' disease, Hashimoto's thyroiditis, autoimmune polyglandular syndrome, insulin-dependent diabetes mellitus, insulin-independent diabetes mellitus, immune-mediated infertility, autoimmune Addison's disease, pemphigus vulgaris, pemphigus foliaceus, dermatitis herpetiformus, vitiligo, autoimmune hemolytic anemia, autoimmune thrombocytopenic purpura, pernicious anemia, myasthenia gravis, multiple sclerosis, Guillain-Barre syndrome, stiff-man syndrome, acute rheumatic fever, sympathetic ophthalmia, Goodpasture's syndrome, systemic lupus erythematosus, rheumatoid arthritis, systemic necrotizing vasculitis, Wegener's granulomatosis, antiphospholipid syndrome, Sjögren's syndrome, scleroderma, accelerated graft arteriosclerosis, graft-versus-host disease, Crohn's disease, and tuberculosis.

8. The method of claim 1, wherein the effective amount of a nitroxide antioxidant is within a range of 0.01-300 mg/kg.

9. The method of claim 1, wherein the effective amount of a nitroxide antioxidant is within a range of 0.1-250 mg/kg.

10. The method of claim 1, wherein the effective amount of a nitroxide antioxidant is within a range of 1-200 mg/kg.

11. The method of claim 1, wherein the effective amount of a nitroxide antioxidant is within a range of 2-150 mg/kg.

12. The method of claim 1, wherein the effective amount of a nitroxide antioxidant is within a range of 5-125 mg/kg.

13. The method of claim 1, wherein the effective amount of a nitroxide antioxidant is within a range of 7-100 mg/kg.

14. The method of claim 1, wherein the effective amount of a nitroxide antioxidant is within a range of 10-75 mg/kg.

15. The method of claim 1, wherein the effective amount of a nitroxide antioxidant is within a range of 15-30 mg/kg.

16. A method for inhibiting the progression of an immunological disease, comprising:
identifying an individual affected by or at risk for the immunological disease; and
administering to that individual an amount of a nitroxide antioxidant effective to alter expression of a gene associated with the immunological disease.

17. The method of claim 16, wherein the expression of the immunological disease-associated proteins is increased.

18. The method of claim 17, wherein the immunological disease-associated protein is glucocorticoid-induced leucine zipper.

19. The method of claim 16, wherein the expression of the immunological disease-associated proteins is decreased.

20. The method of claim 19, wherein the immunological disease-associated protein is selected from the group consisting of collagen type 1A1 and complement component 6.

21. The method of claim 16, wherein the nitroxide antioxidant is 4-hydroxy-2,2,6,6-tetramethylpiperidine-1-oxyl.

22. The method of claim 16, wherein the immunological disease is selected from the group consisting of Graves' disease, Hashimoto's thyroiditis, autoimmune polyglandular syndrome, insulin-dependent diabetes mellitus, insulin-independent diabetes mellitus, immune-mediated infertility, autoimmune Addison's disease, pemphigus vulgaris, pemphigus foliaceus, dermatitis herpetiformus, vitiligo, autoimmune hemolytic anemia, autoimmune thrombocytopenic purpura, pernicious anemia, myasthenia gravis, multiple sclerosis, Guillain-Barre syndrome, stiff-man syndrome, acute rheumatic fever, sympathetic ophthalmia, Goodpasture's syndrome, systemic lupus erythematosus, rheumatoid arthritis, systemic necrotizing vasculitis, Wegener's granulomatosis, antiphospholipid syndrome, Sjögren's syndrome, scleroderma, accelerated graft arteriosclerosis, graft-versus-host disease, Crohn's disease, and tuberculosis.

23. The method of claim 16, wherein the effective amount of the nitroxide antioxidant is within a range of 0.01-300 mg/kg.

24. The method of claim 16, wherein the effective amount of the nitroxide antioxidant is within a range of 0.1-250 mg/kg.

25. The method of claim 16, wherein the effective amount of the nitroxide antioxidant is within a range of 1-200 mg/kg.

26. The method of claim 16, wherein the effective amount of the nitroxide antioxidant is within a range of 2-150 mg/kg.

27. The method of claim 16, wherein the effective amount of the nitroxide antioxidant is within a range of 5-125 mg/kg.

28. The method of claim 16, wherein the effective amount of the nitroxide antioxidant is within a range of 7-100 mg/kg.

29. The method of claim 16, wherein the effective amount of the nitroxide antioxidant is within a range of 10-75 mg/kg.

30. The method of claim 16, wherein the effective amount of the nitroxide antioxidant is within a range of 15-30 mg/kg.

31. A method for treating immunological disease, comprising:
administering to an immunological disease patient an amount of a nitroxide antioxidant effective to inhibit macrophage activity.

32. The method of claim 31, wherein the nitroxide antioxidant is administered in an amount effective to increase intracellular levels of at least one protein related to inhibition of macrophage activity.

33. The method of claim 31, wherein the effective amount of the nitroxide antioxidant is within a range of 0.01-300 mg/kg.

34. The method of claim 31, wherein the effective amount of the nitroxide antioxidant is within a range of 0.1-250 mg/kg.

35. The method of claim 31, wherein the effective amount of the nitroxide antioxidant is within a range of 1-200 mg/kg.

36. The method of claim 31, wherein the effective amount of the nitroxide antioxidant is within a range of 2-250 mg/kg.

37. The method of claim 31, wherein the effective amount of the nitroxide antioxidant is within a range of 5-125 mg/kg.

38. The method of claim 31, wherein the effective amount of the nitroxide antioxidant is within a range of 7-100 mg/kg.

39. The method of claim 31, wherein the effective amount of the nitroxide antioxidant is within a range of 10-75 mg/kg.

40. The method of claim 31, wherein the effective amount of the nitroxide antioxidant is within a range of 15-30 mg/kg.

41. The method of claim 31, wherein the protein related to inhibition of macrophage activity is glucocorticoid-induced leucine zipper.

42. A method for treating immunological disease, comprising:
administering to an immunological disease patient an amount of an nitroxide antioxidant effective to inhibit activity of the membrane attack complex.

43. The method of claim 42, wherein the nitroxide antioxidant is administered in an amount effective to decrease intracellular levels of at least one protein related to inhibition of the activity of the membrane attack complex.

44. The method of claim 42, wherein the effective amount of the nitroxide antioxidant is within a range of 0.01-300 mg/kg.

45. The method of claim 42, wherein the effective amount of the nitroxide antioxidant is within a range of 0.1-250 mg/kg.

46. The method of claim 42, wherein the effective amount of the nitroxide antioxidant is within a range of 1-200 mg/kg.

47. The method of claim 42, wherein the effective amount of the nitroxide antioxidant is within a range of 2-250 mg/kg.

48. The method of claim 42, wherein the effective amount of the nitroxide antioxidant is within a range of 5-125 mg/kg.

49. The method of claim 42, wherein the effective amount of the nitroxide antioxidant is within a range of 7-100 mg/kg.

50. The method of claim 42, wherein the effective amount of the nitroxide antioxidant is within a range of 10-75 mg/kg.

51. The method of claim 42, wherein the effective amount of the nitroxide antioxidant is within a range of 15-30 mg/kg.

52. The method of claim 42, wherein the protein related to inhibition of macrophage activity is complement component 6.

53. Use of a nitroxide antioxidant in the preparation of a medicament for altering intracellular levels of one or more proteins associated with an immunological disease.

54. Use of a nitroxide antioxidant in the preparation of a medicament for inhibiting the progression of an immunological disease.

55. Use of a nitroxide antioxidant in the preparation of a medicament for inhibiting macrophage activity.

56. Use of a nitroxide antioxidant in the preparation of a medicament for inhibiting activity of the membrane attack complex.
